(19) United States
(12) Patent Application Publication
(10) Pub. No.: US 2026/0083792 A1
(43) Pub. Date: Mar. 26, 2026

(54) METHODS AND COMPOSITIONS USING DICHROSTACHYS GLOMERATA TO REDUCE ANXIETY, FOOD CRAVINGS AND STRESS AND IMPROVE MOOD, PHYSICAL HEALTH, MENTAL HEALTH, ENERGY AND HAPPINESS IN A MAMMAL

(71) Applicant: Shil Kothari, Fairfield, CA (US)

(72) Inventor: Shil Kothari, Fairfield, CA (US)

(21) Appl. No.: 18/896,862

(22) Filed: Sep. 25, 2024

(51) Int. Cl. A61K 36/48; A61P 3/04

(52) U.S. Cl. CPC A61K 36/48; A61P 3/04

(57) ABSTRACT

Methods and related compositions using Dichrostachys glomerata to provide a variety of health and wellness benefits in a mammal. More specifically, methods and compositions using Dichrostachys glomerata to reduce anxiety, food cravings, stress and improve mood, physical health, mental health, energy and happiness and improve other quality of life factors in one or more mammals, are provided. In one embodiment, a composition for reducing food cravings in a mammal is provided, wherein the composition comprises an effective amount of Dichrostachys glomerata fruit pods provided as an oral dosage unit in the form of a pill, capsule, liquid, lozenge or tablet.

### FIELD OF THE DISCLOSURE

The present disclosure relates to methods and related compositions using Dichrostachys glomerata to provide a variety of health and wellness benefits to a mammal. More specifically, the present disclosure is directed to methods and compositions for using Dichrostachys glomerata to reduce anxiety, food cravings and stress and improve mood, physical health, mental health, energy, happiness and improve other quality of life factors in one or more mammals.

### BACKGROUND

Dichrostachys glomerata is a semi-deciduous to deciduous tree up to about seven meters tall with an open crown. Bark on young branches appear green and hairy but dark grey-brown and longitudinally fissured on older branches and stems; smooth on spines formed from modified side shoots.

Dichrostachys.glomerata contains various components such as flavonoids, phenolic compounds, alkaloids, tannins, saponins, and terpenoids (Djuissi et al., 2021). Dyglomera is a standardized powder prepared by extracting Dichrostachys glomerata fruit pods with aqueous ethanol followed by concentration and drying. In vitro and in vivo research has shown that Dichrostachys glomerata has antioxidant properties and the ability to lower fasting serum glucose levels and glycated hemoglobin (Etoudi et al., 2010; Kuate et al., 2010, 2011). As well, Dyglomera is thought to have some anti-inflammatory and fat regulation activity in animals and obese and type 2 diabetes patients (Azantsa et al., 2025; Dieudonne et al., 2013; Kuate et al., 2013, Kim et al., 2022). Finally, Dichrostachys glomerata was found safe in rats and genotoxicity tests (Kothari et al., 2014).

The results shown below and in the associated figures determined the effect Dyglomera (Dichrostachys glomerata) has on food cravings, mood, anxiety, stress, physical health, mental health, energy, happiness and various other health-related quality factors in overweight and mildly obese adults, using a randomized double-blind placebo-controlled trial design described and shown below. The results showed an improvement in various factors, including but not limited to, reducing anxiety, food cravings and stress and improving mood, physical health, mental health, energy and happiness and improved health-related quality of life factors, in a mammal.

### SUMMARY OF THE INVENTION

In one embodiment of the present disclosure, a composition for reducing food cravings in a mammal is provided. The composition comprises (in no particular order for any of the embodiments disclosed herein): an effective amount of Dichrostachys glomerata provided as an oral dosage unit in the form of a pill, capsule, liquid, lozenge or tablet.

In another embodiment of the present disclosure, a composition comprising an effective amount of Dichrostachys glomerata fruit pods; provided as an oral dosage unit in the form of a pill, capsule, liquid, lozenge or tablet, is provided.

In yet another embodiment of the present disclosure, a method of reducing food cravings in a mammal, the method comprising providing a composition containing an effective amount of Dichrostachys glomerata to a mammal as an oral dosage unit in the form of a pill, capsule, liquid, lozenge or tablet, is provided.

In yet another embodiment of the present disclosure, the effective amount of Dichrostachys glomerata is 100 mg to 900 mg daily for at least 30 days.

In yet another embodiment of the present disclosure, the effective amount of Dichrostachys glomerata is 100 mg to 900 mg daily.

In yet another embodiment of the present disclosure, the Dichrostachys glomerata composition is an extract of Dichrostachys glomerata fruit.

In yet another embodiment of the present disclosure, the Dichrostachys glomerata composition is an ethanol extract of Dichrostachys glomerata.

In yet another embodiment of the present disclosure, the Dichrostachys glomerata composition is comprised of Dichrostachys glomerata fruit pods.

In yet another embodiment of the present disclosure, the Dichrostachys glomerata composition is comprised of dried Dichrostachys glomerata fruit pods.

In yet another embodiment of the present disclosure, the composition is provided to a mammal for at least 30 days.

### BRIEF DESCRIPTION OF THE DRAWINGS

The foregoing and other objects, aspects, features, and advantages of the present disclosure will become more apparent and better understood by referring to the following description taken in conjunction with the accompanying drawings and/or figures, in which:

FIG. 1 is a table showing Mean (M) and Standard Deviation (SD) Scores for experiments that were performed and the Profile of Mood States, Trait Anxiety, Perceived Stress Scale, Food Cravings, Weight (lbs), and Calories for the Dyglomera (extract of Dichrostachys glomerata fruit) and Placebo Groups. Lower scores indicate an improvement for the Profile of Mood States, Trait Anxiety Inventory, Perceived Stress Scale, Food Cravings Questionnaire, Cortisol Levels, and Weight. †=significant improvement from Baseline; ‡=significant improvement for Dyglomera (extract of Dichrostachys glomerata fruit) Group vs. Placebo Group; §=the Placebo Group consumed less calories than the Dyglomera (extract of Dichrostachys glomerata fruit) Group.

FIG. 2 is a table showing Mean (M) and Standard Deviation (SD) Scores for the Health-related Quality of Life Outcomes for the Dyglomera (extract of Dichrostachys glomerata fruit) and Placebo Groups. Higher scores=improvements for General Health. For all other items, a lower score indicates an improvement. †=significant improvement from Baseline; ‡=significant improvement for Dyglomera (extract of Dichrostachys glomerata fruit) Group vs. Placebo Group.

FIG. 3 is a graph showing Weight Loss (lbs) at Baseline, Day 30, and Day 60 for the Dyglomera (extract of Dichrostachys glomerata fruit) group and Placebo Group. Dyglomera (extract of Dichrostachys glomerata fruit) group lost significantly more weight from Baseline to Day 60 compared to the Placebo Group, p<0.05.

FIG. 4 is a table showing Repeated-Measures ANOVA for Health-Related Quality of Life. Interpretation: Higher scores indicate an improvement in general health. The results show no significant effects for either Time or Interaction. The results demonstrate that the Dyglomera (extract of Dichrostachys glomerata fruit) group experienced a significant improvement in various general health measures and factors shown and described.

FIG. 5 is a table showing Repeated-Measures ANOVA for Health-Related Quality of Life. Interpretation: Lower scores indicate an improvement in physical health improvement. Significant main effects for Condition and Interaction, but no significant effect for Time was evidenced. Physical health scores decreased significantly for the Placebo group when compared to the Dyglomera (extract of Dichrostachys glomerata fruit) group at the Day 30 time point. Dyglomera (extract of Dichrostachys glomerata fruit) group physical health improved from baseline to Day 60 compared to worsening for the Placebo group. #=significant differences from Placebo Group to Dyglomera (extract of Dichrostachys glomerata fruit) group.

FIG. 6 is a table showing Repeated-Measures ANOVA for Health-Related Quality of Life. Interpretation: Lower scores indicate an improvement in mental health. Results show significant main effects for Condition and Interaction. Mental health scores decreased significantly for the Placebo group when compared to the Dyglomera (extract of Dichrostachys glomerata fruit) group at the Day 30 time point. There was also a significant difference from Baseline to Day 30 for the Placebo group. Dyglomera (extract of Dichrostachys glomerata fruit) group mental health improved from baseline to Day 60 compared to the Placebo group. +=significant differences from Baseline to Day 60. #=significant differences from Placebo Group to Dyglomera (extract of Dichrostachys glomerata fruit) Group.

FIG. 7 is a table showing Repeated-Measures ANOVA for Health-Related Quality of Life. Interpretation: Lower scores indicate an improvement. Results show significant main effects for Condition and Time and Interaction. Scores for health deterring activities decreased significantly for the Placebo group when compared to the Dyglomera (extract of Dichrostachys glomerata fruit) group at the Day 30 time point. There was also a significant difference from Baseline to Day 30 for the Placebo group. +=significant differences from Baseline to Day 60. #=significant differences from Placebo Group to Dyglomera (extract of Dichrostachys glomerata fruit) Group.

FIG. 8 is a table showing Repeated-Measures ANOVA for Health-Related Quality of Life. Interpretation: Lower scores indicate an improvement in pain deterring activities. Significant main effects for Condition was evidenced, but not for Time or Interaction. Scores for pain deterring activities significantly improved from Baseline to Day 60 for the Dyglomera (extract of Dichrostachys glomerata fruit) Group and worsened for the Placebo group. +=significant differences from Baseline to Day 60.

FIG. 9 is a table showing Repeated-Measures ANOVA for Health-Related Quality of Life. Interpretation: Lower scores indicate an improvement in feeling sad, blue, or depressed. Results show significant main effects for Time and Condition, but no significant effect for Interaction. Scores decreased significantly from Baseline to Days 30 and 60 for the Placebo group. *=significant differences from Baseline to Day 30. +=significant differences from Baseline to Day 60.

FIG. 10 is a table showing Repeated-Measures ANOVA for Health-Related Quality of Life. Interpretation: Lower scores indicate an improvement in feeling worried, tense, or anxious. Results show significant main effects for Time and Condition and Interaction. Scores decreased significantly from Baseline to Days 30 and 60 for the Placebo group. *=significant differences from Baseline to Day 30. +=significant differences from Baseline to Day 60.

FIG. 11 is a table showing Repeated-Measures ANOVA for Health-Related Quality of Life. Interpretation: Lower scores indicate an improvement in feeling more rested. Results show significant main effects for Time and Condition, but no significant effect for Interaction. Scores decreased significantly from Baseline to Day 60 for the both the Dyglomera (extract of Dichrostachys glomerata fruit) and Placebo groups; and from Baseline to Day 30 for the Placebo Group *=significant differences from Baseline to Day 30. +=significant differences from Baseline to Day 60.

FIG. 12 is a table showing Repeated-Measures ANOVA for Health-Related Quality of Life. Higher scores indicate an improvement in feeling healthy/full of energy. Results show significant main effects for Time, Interaction, and Condition. Scores increased significantly from Baseline to Days 30 and 60 for the Placebo group. The Interaction was at the Baseline time point when Dyglomera (extract of Dichrostachys glomerata fruit) outperformed Placebo group scores. *=significant differences from Baseline to Day 30. +=significant differences from Baseline to Day 60.

FIG. 13 is a table showing Repeated-Measures ANOVA for Profile of Mood States (POMS) Surveys. Interpretation: Lower scores indicate an improvement in tension symptoms. Results show significant main effects for Time, Interaction, or Condition. The Dyglomera (extract of Dichrostachys glomerata fruit) group significantly improved when compared to the Placebo group at the Day 60 time point. There was also a significant difference between Baseline and Day 60 for the Dyglomera (extract of Dichrostachys glomerata fruit) group and Baseline to Day 30 for the Placebo group. *=significant differences from Baseline to Day 30. +=significant differences from Baseline to Day 60. #=significant difference between Dyglomera (extract of Dichrostachys glomerata fruit) and Placebo.

FIG. 14 is a table showing Repeated-Measures ANOVA for Profile of Mood States (POMS) Surveys. Interpretation: Lower scores indicate an improvement in anger symptoms. Results show significant main effect for Condition, but no significant effects for Interaction and Time. Anger scores decreased significantly from Baseline to Day 60 for the Dyglomera (extract of Dichrostachys glomerata fruit) group and Baseline to Day 30 for the Placebo group. *=significant differences from Baseline to Day 30. +=significant differences from Baseline to Day 60.

FIG. 15 is a table showing Repeated-Measures ANOVA for Profile of Mood States (POMS) Surveys. Interpretation: Lower scores indicate an improvement in fatigue symptoms. Results show significant main effects for Condition, Time, and Interaction. Fatigue scores decreased significantly for the Placebo group when compared to the Dyglomera (extract of Dichrostachys glomerata fruit) group at the Day 30 time point. There was also a significant improvement from Baseline to Day 30 for the Placebo group. *=significant differences from Baseline to Day 30. +=significant differences from Baseline to Day 60. #=significant differences from Placebo Group to Dyglomera (extract of Dichrostachys glomerata fruit) Group.

FIG. 16 is a table showing Repeated-Measures ANOVA for Profile of Mood States (POMS) Surveys. Interpretation: Lower scores indicate an improvement in depression symptoms. The results show a significant main effect for Condition, but no significant effects for Time or Interaction.

FIG. 17 is a table showing Repeated-Measures ANOVA for Profile of Mood States (POMS) Surveys. Interpretation: Higher scores indicate an improvement in esteem. Results show significant main effects for Condition, but not Time or Interaction. Scores for esteem significantly improved from Baseline to Day 30 for the Placebo Group. *=significant differences from Baseline to Day 30.

FIG. 18 is a table showing Repeated-Measures ANOVA for Profile of Mood States (POMS) Surveys. Interpretation: Higher scores indicate an improvement in vigor. Results show significant main effects for Time and Condition. Scores increased significantly from Baseline to Day 60 for the Dyglomera (extract of Dichrostachys glomerata fruit) group, and from Baseline to Days 30 and 60 for the Placebo group. There was also a significant difference at the Day 30 time point where the Placebo group outperformed the Dyglomera (extract of Dichrostachys glomerata fruit) group. *=significant differences from Baseline to Day 30. +=significant differences from Baseline to Day 60. #=significant difference between the Dyglomera (extract of Dichrostachys glomerata fruit) and Placebo Group.

FIG. 19 is a table showing Repeated-Measures ANOVA for Profile of Mood States (POMS) Surveys. Interpretation: Lower scores indicate an improvement in confusion. Results show significant main effects for Condition, but no significant effects for Time or Interaction. Scores decreased significantly from Baseline to Day 60 for the Dyglomera (extract of Dichrostachys glomerata fruit) group. +=significant differences from Baseline to Day 60.

FIG. 20 is a table showing Repeated-Measures ANOVA for Profile of Mood States (POMS) Surveys. Interpretation: Lower scores indicate an improvement in total mood scores. Results show significant main effects for Time, Condition, and Interaction. Scores decreased significantly from Baseline to Day 60 for the Dyglomera (extract of Dichrostachys glomerata fruit) group. There was also a significant difference at the Day 60 time point with the Dyglomera (extract of Dichrostachys glomerata fruit) group outperforming the Placebo group. +=significant differences from Baseline to Day 60. #=significant difference between the Dyglomera (extract of Dichrostachys glomerata fruit) and Placebo Group.

FIG. 21 is a table showing Repeated-Measures ANOVA for State Trait Anxiety (STAI) Surveys. Interpretation: Lower scores indicate an improvement in anxiety symptoms. Results show significant main effects for Time and Condition, but not for Interaction. Scores decreased significantly from Baseline to Day 60 for the Dyglomera (extract of Dichrostachys glomerata fruit) group and from Baseline to Days 30 and 60 for the Placebo group. *=significant differences from Baseline to Day 30. +=significant differences from Baseline to Day 60.

FIG. 22 is a table showing Repeated-Measures ANOVA for Perceived Stress (PSS) Surveys. Interpretation: Lower scores indicate an improvement in perceived stress. Results show significant main effect for Condition was evidenced, but not for Time or the Interaction. Scores decreased significantly from Baseline to Day 60 for the Dyglomera (extract of Dichrostachys glomerata fruit) group. +=significant differences from Baseline to Day 60.

FIG. 23 is a table showing Descriptive Statistics Baseline to Week 4 for Weight, Mood, Happiness, Energy, Productivity, Mental Clarity, Cravings, and Physical Activity Daily Diary Survey Results. For self-report assessments higher scores indicate an improvement, except for stress, mood, happiness, energy, weight, and food cravings where a lower score indicates an improvement.

FIG. 24 is a table showing Descriptive Statistics Weeks 5 to 8 for Weight, Mood, Happiness, Energy, Productivity, Mental Clarity, Cravings, and Physical Activity Daily Diary Surveys Results.

FIG. 25 is a table showing Repeated-Measures ANOVAs Mood Daily Diary Surveys. Interpretation: Lower scores indicate an improvement. Pair wise comparisons show that there were significant differences from Baseline to Weeks 2 & 4, Week 1 to Weeks 2 & 4, Week 2 to Week 5 & 6, Week 3 to Week 4, and Week 4 to Weeks 5-8 for the Placebo group. There was also a significant difference at Week 4 where the Placebo group outperformed the Dyglomera (extract of Dichrostachys glomerata fruit). #=significant difference between Dyglomera (extract of Dichrostachys glomerata fruit) group and Placebo group.

FIG. 26 is a table showing Repeated-Measures ANOVAs Happiness Daily Diary Surveys. Interpretation: Lower scores indicate an improvement. Pair wise comparisons show that there were significant differences from Baseline to Week 3 and Week 3 to Week 4 for the Dyglomera group. There were significant differences from Baseline to Weeks 2, 4, & 7, Week 1 to Week 4, Weeks 2 to Weeks 4 & 6, Week 3 to Week 4, Week 4 to Weeks 5, 6, & 8, and Week 6 to Week 7 for the Placebo group. There were also significant differences at Week 4 between the Dyglomera (extract of Dichrostachys glomerata fruit) group and the Placebo group, and at Weeks 3, 5, & 6.#=significant difference between Dyglomera (extract of Dichrostachys glomerata fruit) group and Placebo group.

FIG. 27 is a table showing Repeated-Measures ANOVAs Energy Daily Diary Surveys. Interpretation: Lower scores indicate an improvement. Pair wise comparisons show that there were significant differences from Week 1 to Weeks 6 & 8 for the Dyglomera (extract of Dichrostachys glomerata fruit) group. There were significant differences from Baseline to Weeks 5, 7, & 8, Week 1 to Weeks 5-8, Weeks 2 to Weeks 4, 7, & 8, Week 3 to Week 4, and Week 4 to Weeks 5-8 for the Placebo group. There was also a significant difference at Week 4 where the Dyglomera (extract of Dichrostachys glomerata fruit) group score was significantly different from the Placebo group. #=significant difference between Dyglomera (extract of Dichrostachys glomerata fruit) group and Placebo group.

FIG. 28 is a table showing Repeated-Measures ANOVAs Productivity Daily Diary Surveys. Interpretation: Higher scores indicate an improvement. Pair wise comparisons show that there was a significant difference from Week 3 to Week 8 for the Dyglomera (extract of Dichrostachys glomerata fruit) group. There were significant differences from Week 3 to Week 5, and Week 5 to Weeks 7 & 8 for the Placebo group. There were also significant differences at Weeks 2, 3, 6-8 where the Placebo group outperformed the Dyglomera (extract of Dichrostachys glomerata fruit) group. #=significant difference between Placebo group and the Dyglomera (extract of Dichrostachys glomerata fruit) group.

FIG. 29 is a table showing Repeated-Measures ANOVAs Stress Daily Diary Surveys. Interpretation: Lower scores indicate an improvement. Pair wise comparisons show that there was a significant difference from Baseline to Weeks 1-4, 7, & 8, and Week 5 to Week 7 for the Dyglomera (extract of Dichrostachys glomerata fruit) group. There were significant differences from Baseline to Week 8, Week 1 to Weeks 4 & 6, Week 2 to Week 8, Week 3 to Week 8, Week 4 to Weeks 5 & 8, Week 6 to Week 8, and Week 7 to Week 8 for the Placebo group. There were also significant differences at Weeks 2, 3, 4, 6, & 7 where the Dyglomera (extract of Dichrostachys glomerata fruit) group outperformed the Placebo group. #=significant difference between Dyglomera (extract of Dichrostachys glomerata fruit) group and Placebo group.

FIG. 30 is a table showing Repeated-Measures ANOVAs Mental Clarity Daily Diary Surveys. Interpretation: Higher scores indicate an improvement. Pair wise comparisons show that there was a significant difference from Baseline to Weeks 6 & 8, Week 1 to Weeks 6 & 8, Week 2 to Weeks 6 & 8, Week 3 to Weeks 6 & 8, Week 4 to Week 8, and Week 5 to Weeks 6 & 8 for the Dyglomera (extract of Dichrostachys glomerata fruit) group. There were significant differences from Week 2 to Weeks 5 & 6 for the Placebo group. There were also significant differences at Weeks 3 & 5 where the Placebo group outperformed the Dyglomera (extract of Dichrostachys glomerata fruit) group. #=significant difference between Placebo group and the Dyglomera (extract of Dichrostachys glomerata fruit) group.

FIG. 31 is a table showing Repeated-Measures ANOVAs Cravings Daily Diary Surveys. Interpretation: Lower scores indicate an improvement. Pair wise comparisons show that there was a significant difference from Baseline to Weeks 1, 2, 4, 6, & 8, and Week 3 to Week 8 for the Dyglomera (extract of Dichrostachys glomerata fruit) group. There were significant differences from Baseline to Week 7, Week 1 to Weeks 2, 3, 4, & 6, Week 3 to Week 7, Week 4 to Week 7, Week 5 to Week 7, Week 6 to Week 7, and Week 7 to Week 8 for the Placebo group. There were also significant differences at Weeks 2 & 7 where the Dyglomera (extract of Dichrostachys glomerata fruit) group outperformed the Placebo group. #=significant difference between Dyglomera (extract of Dichrostachys glomerata fruit) group and Placebo group.

FIG. 32 is a table showing Repeated-Measures ANOVAs Physical Activity Daily Diary Surveys. Interpretation: Higher scores indicate an improvement. Pair wise comparisons show that there was a significant difference from Baseline to Week 8, Week 1 to Weeks 6 & 8, Week 2 to Week 8, Week 3 to Week 4, Week 4 to Weeks 6 & 8, Week 5 to Week 8, and Week 7 to Week 8 for the Placebo group. There were also significant differences at Weeks 1 & 4 where the Dyglomera (extract of Dichrostachys glomerata fruit) group outperformed the Placebo group. #=significant difference between Dyglomera (extract of Dichrostachys glomerata fruit) group and Placebo group.

FIG. 33 is a table showing Repeated-Measures ANOVAs Anxiety Daily Diary Surveys. Interpretation: Lower scores indicate an improvement. Pair wise comparisons show that there was a significant difference from Baseline to Weeks 2, 4, & 6-8, Week 2 to Week 5, Week 3 to Week 7, Week 4 to Week 5, and Week 5 to Weeks 6-8 for the Dyglomera group. There were significant differences from Baseline to Week 8, Week 1 to Weeks 2, 3, 4, 6 & 7, Week 2 to Weeks 5 & 8, Week 3 to Weeks 5 & 8, Week 4 to Week 8, Week 5 to Week 7, Week 6 to Week 8, and Week 7 to Week 8 for the Placebo group. There were also significant differences at Weeks 2, 4, & 7 where the Dyglomera (extract of Dichrostachys glomerata fruit) group outperformed the Placebo group and Week 5 where the Placebo group outperformed the Dyglomera (extract of Dichrostachys glomerata fruit) group. #=significant difference between Dyglomera (extract of Dichrostachys glomerata fruit) group and Placebo group.

FIG. 34 is a table showing Descriptive Statistics and Repeated-Measures ANOVA for Calories Daily Diary Surveys. No significant results were evidenced for Time or Interaction. The placebo group consumed less calories than the Dyglomera group.

FIG. 35 is a table showing Physical Activity Descriptions and Repeated-Measures ANOVA. Interpretation: Higher scores indicate an improvement. The results show significant main effects for Time, Interaction, and Condition. There was a significant difference at the Day 60 time point with the Dyglomera (extract of Dichrostachys glomerata fruit) group outperforming the Placebo Group. There was also a significant improvement from Baseline to Day 60 for the Dyglomera (extract of Dichrostachys glomerata fruit) group, and from Baseline to Day 30 and Day 30 to Day 60 in the Placebo group. *=significant differences from Baseline to Day 30. +=significant differences from Baseline to Day 60. #=significant differences between the Dyglomera (extract of Dichrostachys glomerata fruit) and Placebo groups. \hat{ }=significant differences from Day 30 to Day 60.

FIG. 36 is a table showing Physical Activity Descriptions and Repeated-Measures ANOVA. Interpretation: Higher scores indicate better exercise habits. Results show significant main effects for Condition, but no significant effect for Time or Interaction. Exercise habit scores increased significantly for the Dyglomera (extract of Dichrostachys glomerata fruit) group from Baseline to Day 60. +=significant differences from Baseline to Day 60.

FIG. 37 is a table showing Physical Activity Descriptions and Repeated-Measures ANOVA. Interpretation: Higher scores indicate improvement. No significant results were evidenced for Time or Interaction. Activity increased from Baseline to Day 30 for the Placebo Group. *=significant differences from Baseline to Day 30.

FIG. 38 is a table showing Physical Activity Descriptions and Repeated-Measures ANOVA. Interpretation: Higher scores indicate more exertion. The results show significant main effects for Condition and Time, but not for Interaction. Scores for exertion increased significantly for the Dyglomera (extract of Dichrostachys glomerata fruit) group from Baseline to Days 30 and 60. *=significant differences from Baseline to Day 30. +=significant differences from Baseline to Day 60.

FIG. 39 is a table showing Physical Activity Descriptions and Repeated-Measures ANOVA. Interpretation: Higher scores indicate higher levels of vigorous activity. Results show significant main effects for Condition and Time, but not for Interaction. Scores for vigorous activity increased significantly for the both the Dyglomera (extract of Dichrostachys glomerata fruit) and Placebo groups from Baseline to Days 30 and 60. *=significant differences from Baseline to Day 30. +=significant differences from Baseline to Day 60.

FIG. 40 is a table showing Physical Activity Descriptions and Repeated-Measures ANOVA. Interpretation: Higher scores indicate improvement. Results show no significant improvement for Time or Interaction.

FIG. 41 is a table showing Physical Activity Descriptions and Repeated-Measures ANOVA. Interpretation: Higher scores indicate an improvement in light activity levels. Results show significant main effects for Time and Condition, but no significant effect for Interaction. Light activity scores increased significantly from Baseline to Days 30 and 60 for the Placebo group. *=significant differences from Baseline to Day 30. +=significant differences from Baseline to Day 60.

FIG. 42 is a table showing Physical Activity Descriptions and Repeated-Measures ANOVA. Interpretation: Lower scores indicate improvement. The results show no significant improvement for Time or Interaction.

FIG. 43 is a table showing Physical Activity Descriptions and Repeated-Measures ANOVA. Interpretation: Lower scores indicate improvement. The results show no significant improvement for Time, or Interaction.

FIG. 44 is a table showing Physical Activity Descriptions and Repeated-Measures ANOVA Cravings Surveys. Interpretation: Lower scores indicate an improvement in craving symptoms. The results show significant main effects for Time, Interaction, and Condition. Scores decreased significantly from Baseline to Day 60 for the Dyglomera (extract of Dichrostachys glomerata fruit) group, and from Baseline to Days 30 and 60 for the Placebo group. The Interaction was at the Day 30 time point when the Placebo group outperformed the Dyglomera (extract of Dichrostachys glomerata fruit) group scores. *=significant differences from Baseline to Day 30. +=significant differences from Baseline to Day 60. #=significant differences between the Dyglomera (extract of Dichrostachys glomerata fruit) and Placebo groups.

FIG. 45 is a table showing Physical Activity Descriptions and Repeated-Measures ANOVA Waist Circumference Surveys. Interpretation: Lower scores indicate an improvement in waist circumference. The results show no significant improvement for Time or Interaction.

FIG. 46 is a table showing Physical Activity Descriptions and Repeated-Measures ANOVA Hip Circumference Surveys. Interpretation: Lower scores indicate an improvement in hip circumference. Results show no significant improvement for Time or Interaction.

FIG. 47 is a table showing Physical Activity Descriptions and Repeated-Measures ANOVA Calories Surveys. Interpretation: The results show no significant improvement for Time or Interaction.

FIG. 48 shows the statistical data of the participant's demographics that participated in the studies shown and described herein.

FIG. 49 is a table showing Descriptive Statistics Cortisol Data.

FIG. 50 is a table showing Repeated-Measures ANOVAs Cortisol Data. Interpretation: The results show no significant time or interaction differences.

### DETAILED DESCRIPTION OF THE INVENTION

The following description is not to be taken in a limiting sense, but is made merely for the purpose of illustrating the general principles of the invention. Various inventive features are described below that can each be used independently of one another or in combination with other features. Various inventive aspects of the compositions, method and related features are described below that can each be used independently of one another or in combination with other features of the methods and compositions disclosed herein.

### Methods

Extensive experiments as described in detail herein and in the figures were performed to determine Dichrostachys glomerata effects on anxiety, food cravings, stress, mood, physical health, mental health, energy and happiness and various other quality of life factors, in one or more mammals. The following is a detailed explanation of the experiments performed, the results of which are shown and described herein and in the figures.
Participants: Participants were 56 overweight and mildly obese adults (M age=44.50, age range=25 to 60 years, n=43 women; BMI M=31.66).
Inclusion criteria: Overweight and mildly obese adults (BMI between 25.00 to 34.99) were selected because they represent the largest proportion of overweight/obese and are the most likely to benefit from a natural weight loss intervention. Globally, more than 40% of the adult population is overweight (BMI of 25 to 29.9) or mildly obese (BMI of 30 to 34.9; CDC, 2021).
Exclusion Criteria: Individuals meeting any of the following criteria were excluded from participation: (1) any metabolic or endocrine related dysregulation including but not limited to: diagnosis of type I or type II diabetes, liver, kidney, or pancreatic dysfunction; (2) history of sleep-affecting disorders; (3) recent highly stressful events within 4 weeks of baseline; (4) usage of weight-influencing medications within 1 month of baseline; (5) use of Ca channel blockers, anxiolytics or SSRIs, no more than 5 times per month, and not within seven days of baseline; (6) unstable use of other medication; (7) current hormone therapy; (8) excessive alcohol consumption; (9) smoking; (10) elevated caffeine intake; (11) irregular sleep-inducing work schedules; (12) inability to engage in spontaneous physical activity; (13) metabolic disorder, a sleep disorder, or a psychiatric condition; (14) pregnancy, attempts at conception, or breastfeeding; (15) use of sleep/weight supplements or medications; (16) actively intermittent fasting, are actively trying to lose weight, or have lost more than ±3 kg in previous 3 months; and (17) individuals deemed incompatible with the study protocol.
Study design: The study described and shown herein was approved by Sterling Institutional Review Board (10504-HAHausenblas) in compliance with the Declaration of Helsinki standards for ethical principles regarding human participant research and registered with ISRCTN registry is ISRCTN70584524.

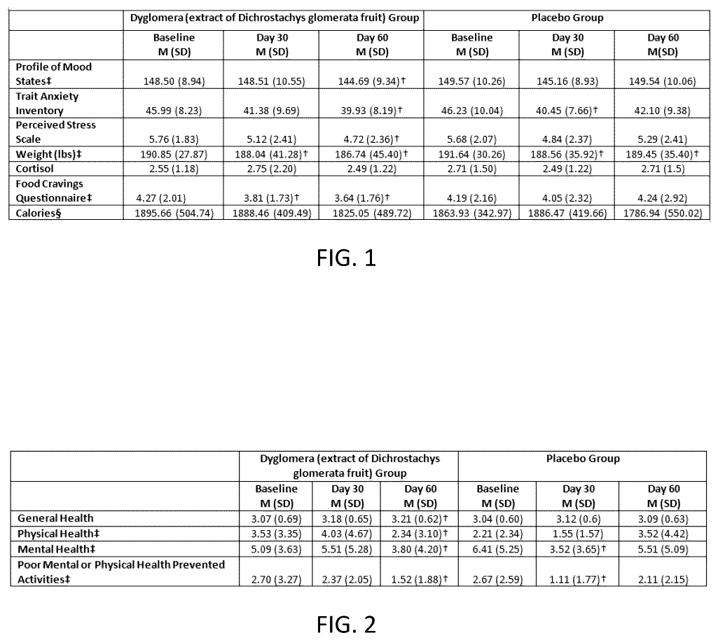

The study was conducted in a double-blind, parallel treatment, stratified random, placebo-controlled manner. The independent variable was the Dichrostachys glomerata nutritional supplementation. The dependent variables were body weight, food cravings, mood, anxiety, stress, and health-related quality of life. Sample size power calculation indicated that 35 participants were needed in each group to achieve a power of 80% and alpha<0.05 (https://clincalc.com/stats/samplesize.aspx).
Procedures: Following preliminary screening, eligible participants provided Institutional Review Board approved informed consent prior to enrollment. Participants completed psychometrically validate self-report questionnaires on Day 0 (Pre), Day 30, and Day 60. In addition, participants maintained a daily diary to document adherence and adverse events. Participants completed the self-report surveys via a Survey Monkey link that was sent via email or text. Completion of the surveys took about 25 minutes at each assessment. Participants were instructed to maintain their habitual lifestyle patterns and refrain from introducing new exercise, diet, or health interventions during the study. These data were collected from March 2023 to June 2023 and were stored electronically.
Intervention: A randomized double-blind placebo-controlled trial design was employed, with participants randomly assigned to either the Dichrostachys glomerata group (DG) or Placebo Control Group (PC or PG) for the duration of the two-month trial. A computer-based randomization via SPSS was used to automate the random assignment process. Participants were directed to consume 300 mg, 1 per day of the allocated substance. Dyglomera, an aqueous ethanol extract of Dichrostachys glomerata fruit pods (standardized to Myricetin 1.6% and Luteolin 1.0%), was supplied by Gateway Health Alliances, Inc (https://www.ghainc.com/; Fairfield, CA, USA). The manufacturing process was as follows: Dichrostachys glomerata fruit pods were extracted using aqueous ethanol. The resulting solution was concentrated and dried to yield Dyglomera. The placebo was rice protein.
Lifestyle: Participants maintained about a 2,000-calorie daily diet throughout the study. Food intake and calorie content were monitored via a 3-Day Food log. Analyses indicated that the calories consumed did not change during the study by time, p's >05.
Adverse Events: The supplement was well-tolerated and only one adverse event was reported for the DG compared to two adverse events reported for the control group (hereafter, “CG”). For the DG, the adverse event reported was gastrointestinal symptoms after taking the supplement. For the CG, the two adverse events reported were heart palpitations and a liver concern.
Trial Reporting: The Consolidated Standards of Reporting Trials (CONSORT, including reporting of harms) was used to report this trial.
Blinding: To ensure that all subjects and researchers were unaware of the treatment assignments, Gateway Health Alliances labeled the supplement/placebo bottles as either A or B. The supplement/placebo pills were identical in color, odor, and size. The research team was blinded to the content of the bottles. At the conclusion of the study, immediately following the last assessment, the research team was unblinded. The participants were then unblinded and informed of their assigned condition.
Supplement Information and Adherence: Participants were instructed to take the capsules about 30 prior to a meal. The participants provided the number of pills remaining in their bottles as an adherence measure. The participants also received a daily text reminder to take their supplement.
Adherence: N=61 participants enrolled and consented and 56 completed the trial, representing an adherence rate of 92%. This adherence rate included the two dropouts from the CG and the 3 participants who withdrew because of adverse events. The reasons for dropout were getting a doctor prescribed weight loss drug during the study (n=1), and personal reasons not related to the study (n=1; see FIG. 1).
Statistical analysis: Data was analyzed for normality by examining skewness and kurtosis scores and using Shapiro-Wilk test and Q-Q plot. Outliers were characterized as data points that exceeded three interquartile ranges beyond 25th and 75th percentiles. However, no extreme outliers were observed. Continuous data were presented as Mean (SD) and analyzed using 2(group)×3 (Time: Baseline, Day 30, Day 60) repeated measures ANOVA. Multiple comparisons were corrected using Sidak Adjustment. Pos hoc tests were paired-sample t-tests where applicable. Categorical variables were analyzed using Chi-square test and expressed as counts/percentage where appropriate. Statistical analyses were performed using Excel and Statistical Product and Service Solutions (SPSS) [version 28].

### Measures

The assessment tools utilized were as follows:
Profile of mood states (POMS) Questionnaire: the POMS-40 was used to assess the mood states of tension, anger, vigor, fatigue, depression, and confusion. A composite score was computed by summing each of the individual scores for tension, depression, anxiety, fatigue, and confusion, with vigor scores subtracted to indicate patients'total mood disturbance. Each item of the POMS items was scored on a 5-point Likert scale ranging from 0 (not at all) to 4 (extremely) with lower scores indicated an improved mood. This scale has good to excellent reliability and validity (McNair et al., 1992).
Trait Anxiety Inventory: The Trait Anxiety Inventory (20-items) was used to measure general feelings of anxiety including general states of calmness, confidence, and security (Spielberger et al., 1983). Higher scores indicate more severe anxiety levels. Each item was assessed on a 4-point Likert-type scale (from 0 to 3 points).
Weight: Participants weight was recorded by a smart scale (BodyTrace, Inc.), which sent participant weights directly to BodyTrace servers via the cellular network. BodyTrace scales have stated accuracy to 0.1 kg and have excellent concordance with weights measured during in-person assessment visits (Ross & Wing, 2016). Weights from these scales were downloaded following the end of the intervention. Participants self-reported weights were also collected via the intervention web server. Weights from both the smart scale and the study website were collected in lbs. Research supports excellent agreement between weights measured via smart scales (i.e., electronic scales), self-report, and objective measures (Krukowski & Ross, 2020).
Dietary Assessments: Nutrition was assessed by a weekly 3-day diet record. Participants recorded all food and beverage intakes for 3 full days (i.e., 2 weekdays, 1 weekend day). The data were inputted into a nutrient analysis software and a report was compiled for total dietary analysis and pertinent macro and micronutrient information.
Food Cravings Questionnaire: The Food Cravings Questionnaire was used to assess food craving. The items are based on a 6-point Likert-type scale ranging from 1 (never/not applicable) to 6 (always). The questionnaire assesses food cravings such as planning to eat food, positive reinforcement from eating, relief from negative mood by eating, lack of control on overeating of food, thoughts about food, physiological state, emotions that are involved during food cravings or eating, environmental cues that may trigger food cravings, and guilt experienced due to food craving. Higher scores indicate greater food cravings. Scores on the Food Cravings Questionnaire have been found to be positively associated with eating pathology, body mass index (BMI), low dieting success, and increases in state food craving during cognitive tasks involving appealing food stimuli (Meule et al., 2014).
Health-related Quality of Life: The CDC Health-related Quality of Life Core Healthy Days measure was used to assess health-related quality of life (CDCP, 2020). The Core Healthy Days module contains one item that asks respondents to rate their general health on a 5-point scale (1=excellent; 3=good; 5=poor). The module also has three items that ask respondents how many days their physical health was not good, their mental health was not good, and their health interfered with their daily activities in the past month (scores can range from 0 days to 30 days). This scale has excellent psychometric properties (Zack et al., 2013).
Perceived Stress Scale: The Perceived Stress Scale-4 measures the degree to which individuals appraise situations in their lives as stressful (Cohen et al., 1983). Specifically, the scale evaluates the degree to which individuals believe their life has been unpredictable, uncontrollable, and overloaded during the previous month. The items are general in nature rather than focusing on specific events or experiences. The scale consisted of four items, and each item was scored on a 5-point Likert scale ranging from 0 (“never”) to 4 (“very often”) with higher scores indicating more perceived stress. This scale has excellent psychometric properties (Du et al., 2023).
Salivary Cortisol: To assess cortisol levels the subjects had their saliva collected in the morning after an overnight fast (i.e., 8 hours). The participants were not engaged in exercise prior to their assessment or consumed any food three hours before the assessment. Synthetic swabs were placed in the oral cavity and chewed for 30 seconds for saliva collection. Saliva samples were obtained by Lets Get Checked synthetic swabs.
Daily diary: The daily diary assessed adverse events and adherence.

### Results

### Weight and Food Cravings

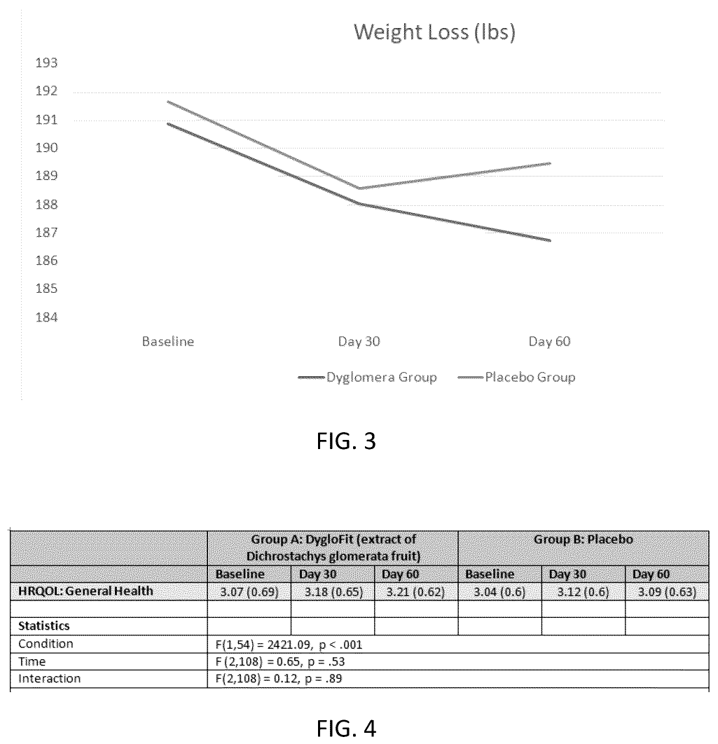

For weight loss, a significant main effect for Condition, F(1,54)=9632.51, p<0.001, and interaction, F(2,218)=2.79, p=0.05, but no significant main effect for time were evidenced, F(2,218)=0.69, p=0.50. DG lost significantly more weight at Day 60 compared to PG. DG lost 4.11 lbs at Day 60 compared to 2.19 lbs for PG (see FIG. 3). For calories consumed a significant main effect for Condition, F(1,54)=5912.15, p<0.001, but not time, F(2,108)=1.32, p=0.27, or interaction, F(2,108)=0.34, p=0.71, were evidenced. PG consumed significantly less calories than DG group, p<0.05 (see FIG. 34).

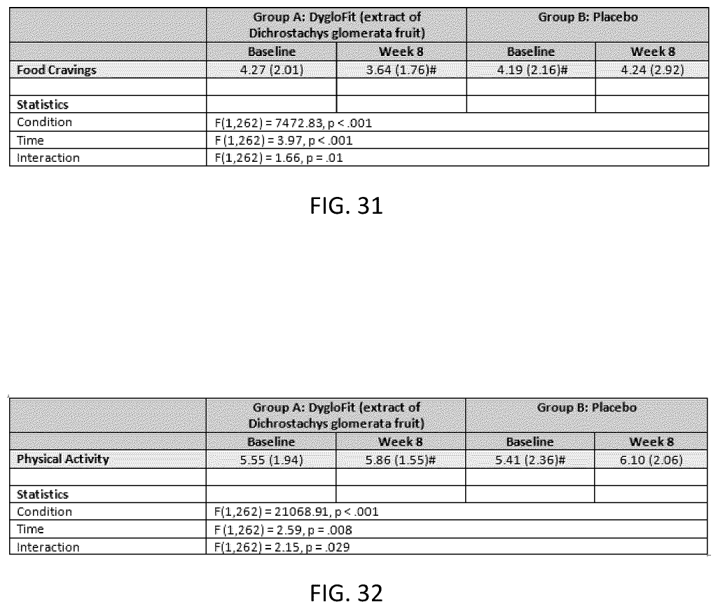

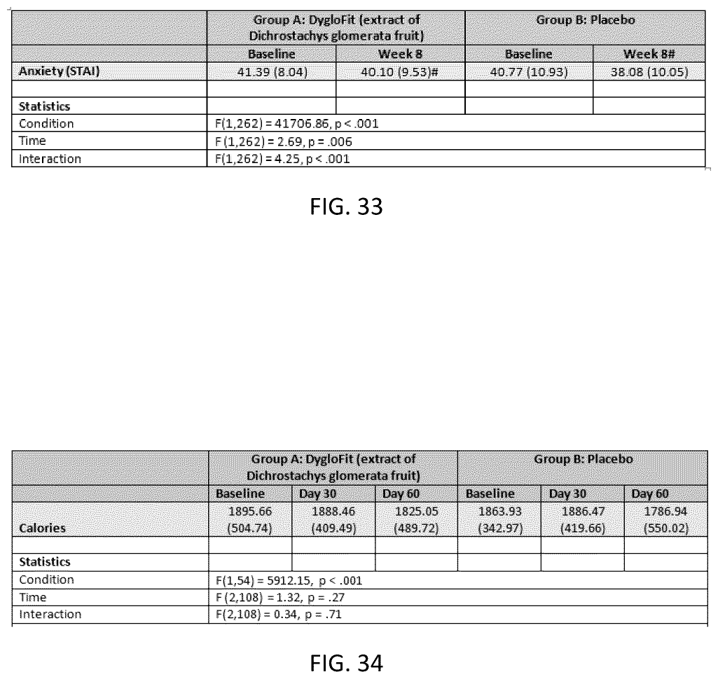

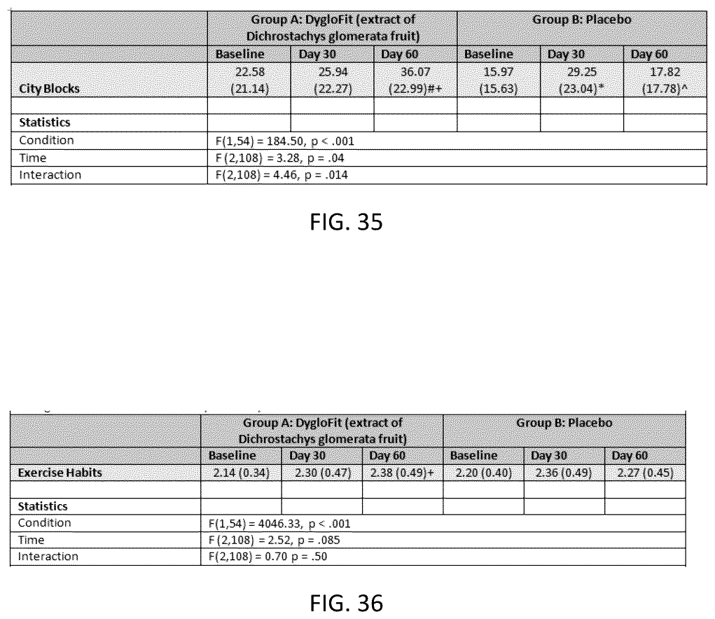

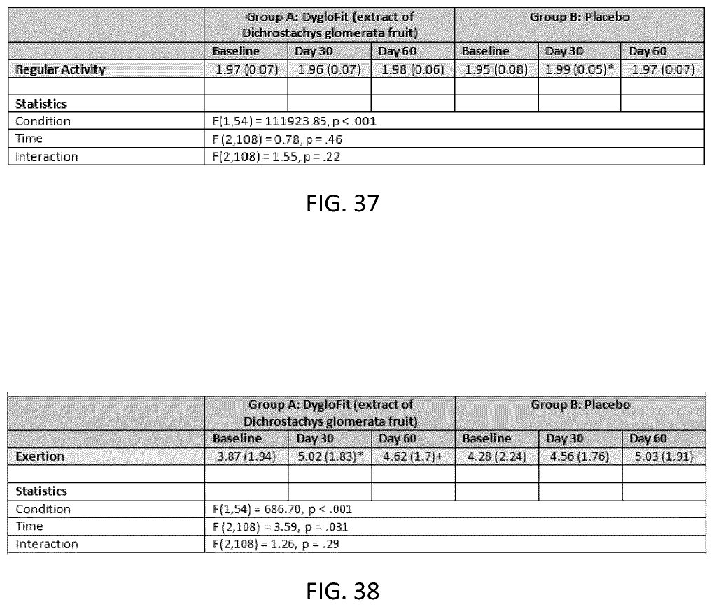

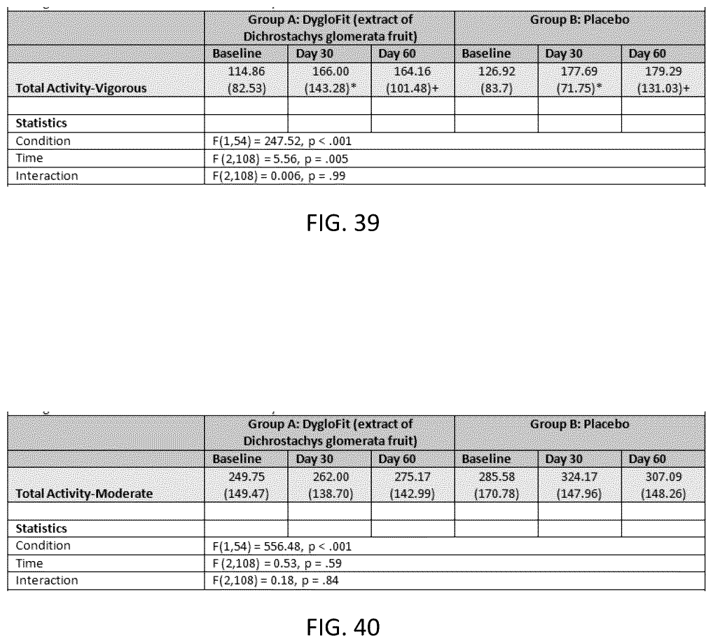

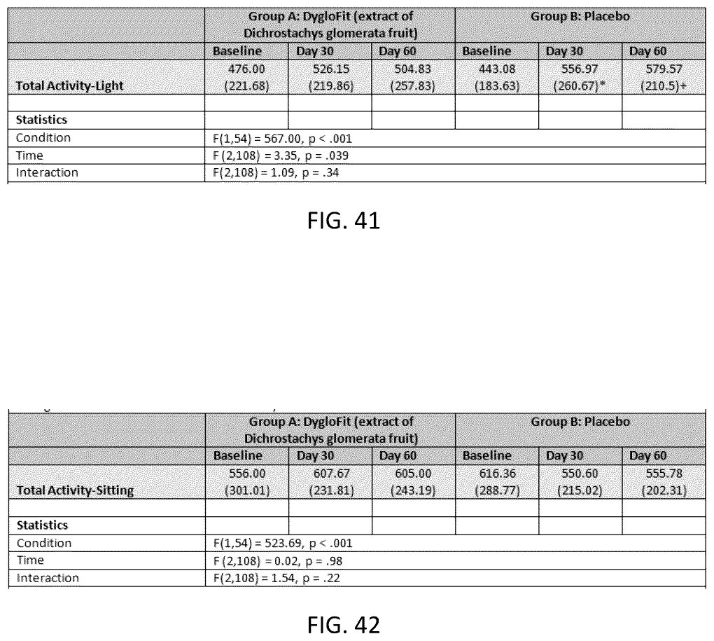

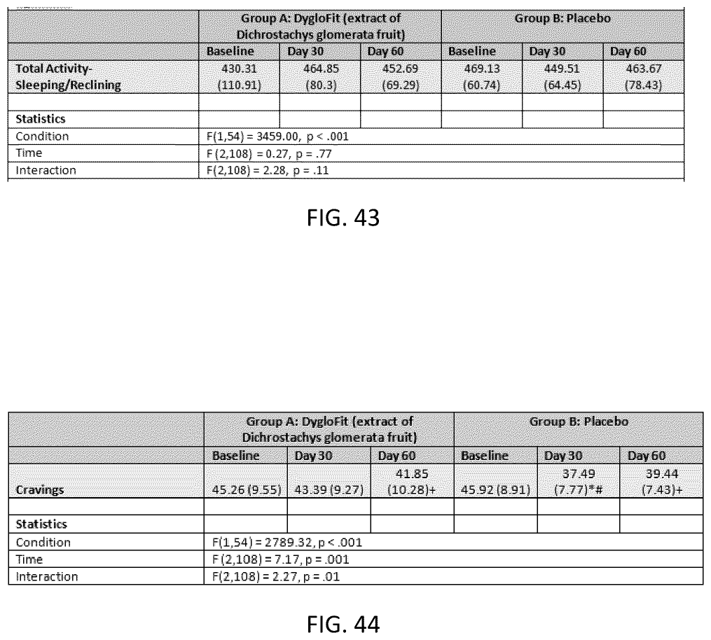

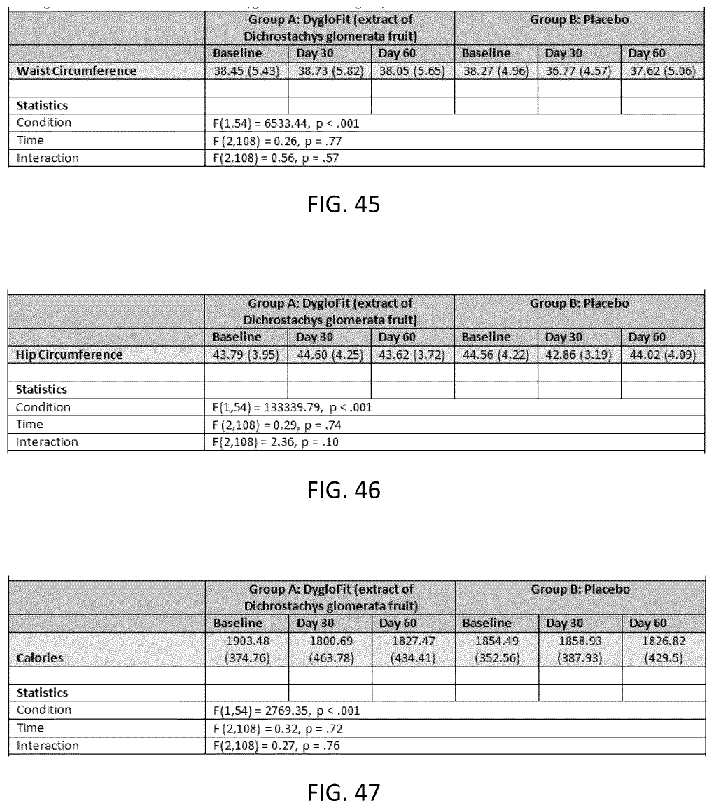

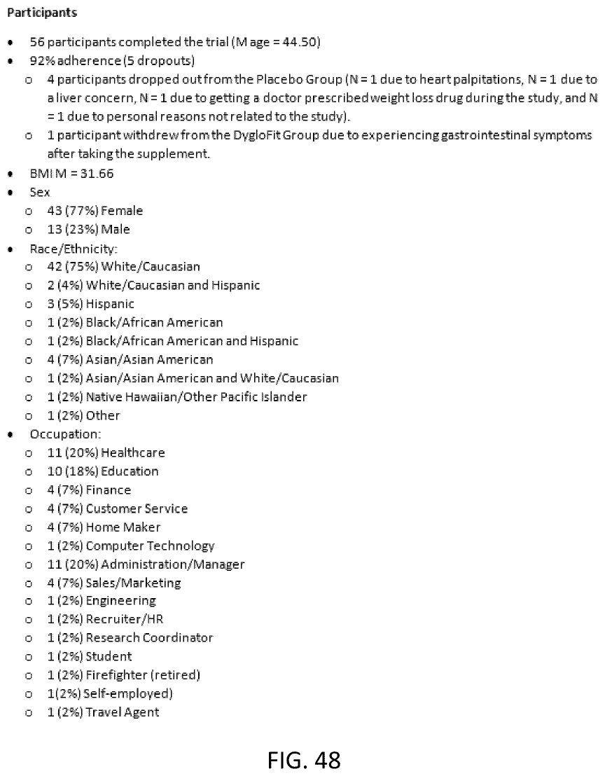

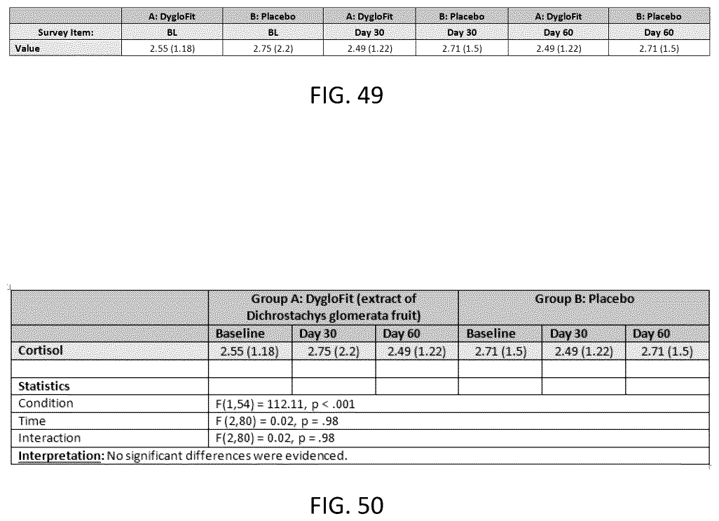

For the Food Cravings, significant Condition, F(1,262)=7472.83, p<0.001, Time, F(1,262)=3.97, p<0.001, and Interaction were evidenced, F(1,262)=1.66, p=0.01. DG had significant reductions in food cravings from Baseline to Day 30 and Day 60 compared to PC (see FIG. 31).

### Mood, Anxiety, and Perceived Stress

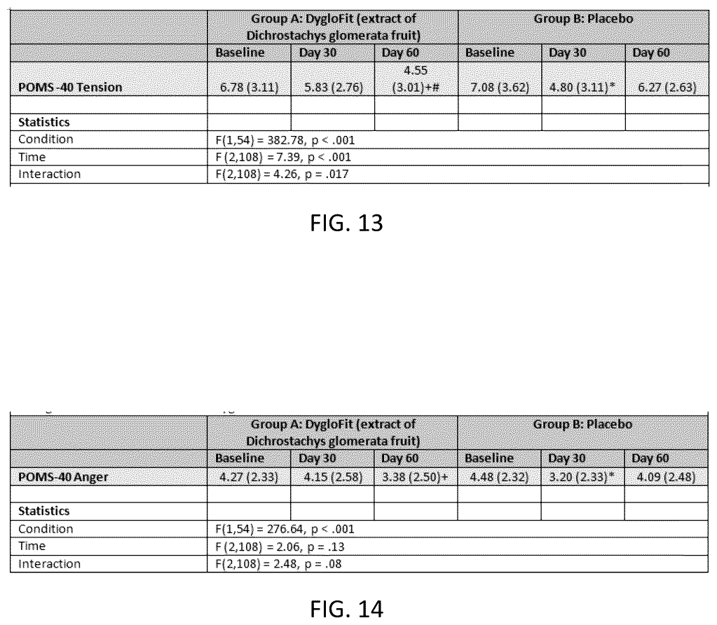

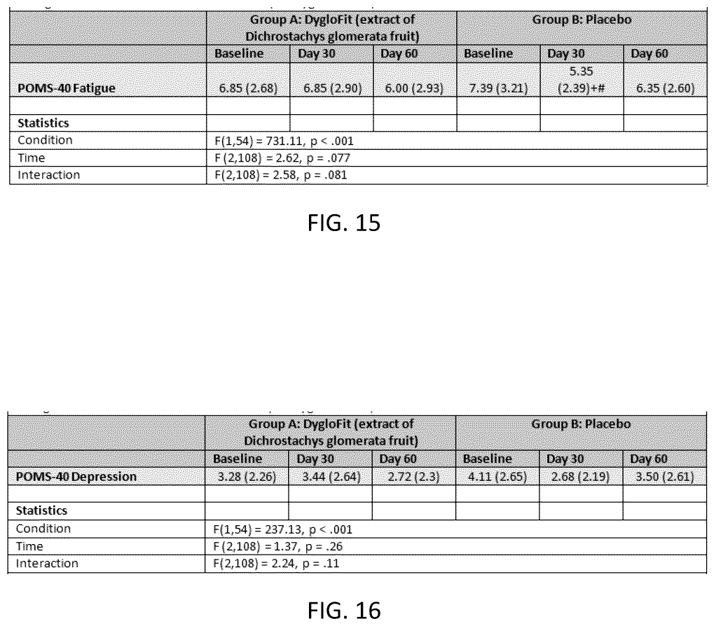

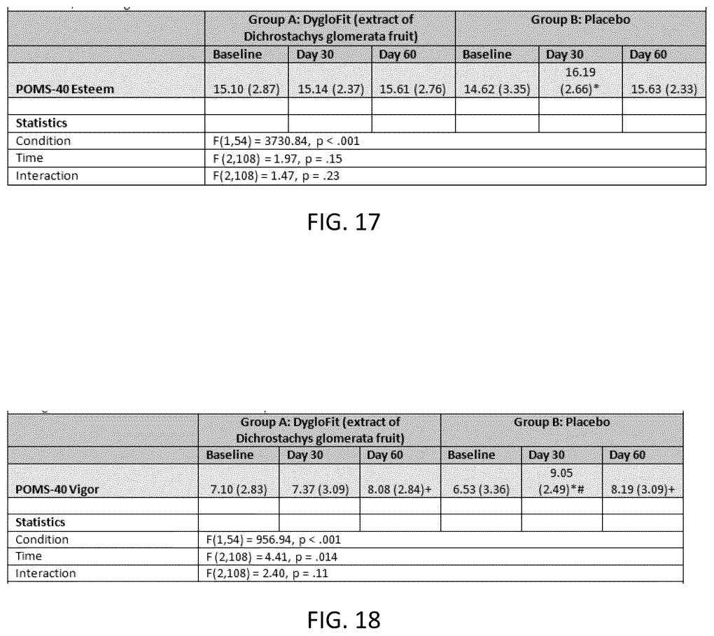

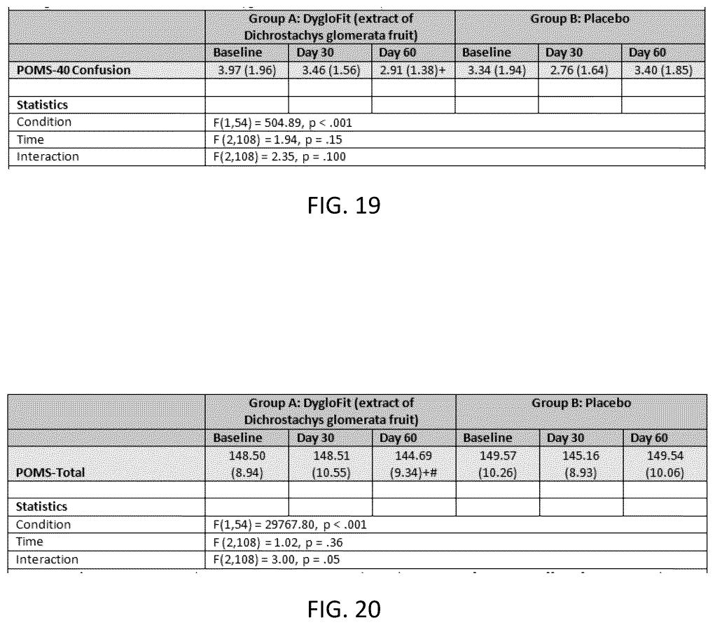

For tension, a significant main effect for Condition, F(1,54)=382.78, p<0.001, and Time, F(2,108)=7.39, p<0.001, as well as a significant Interaction, F(2,108)=4.26, p=0.017, were evidenced. The POMS scores improved significantly from Baseline to Day 60 for DG compared to PG (see FIG. 13).

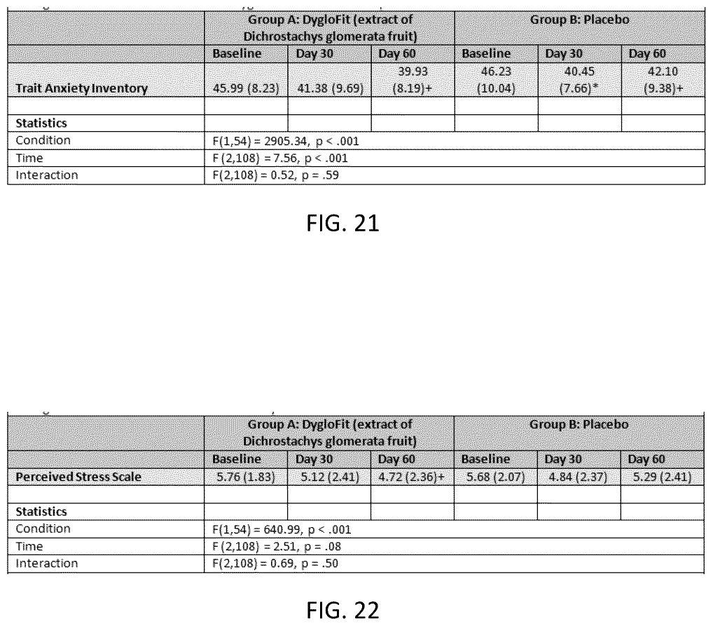

For anxiety, significant main effects for Time, F (2,108)=7.56, p<0.001, and Condition, F(1,54)=2905.34, p<0.001, were evidenced. The interaction was not significant, F(2,108)=0.52, p=0.59. Anxiety decreased significantly from Baseline to Day 60 for DG and from Baseline to Day 30 PG. DG also had a larger improvement in anxiety from Baseline to Day 60 compared to PG, albeit not significant (see FIG. 21).

For the Perceived Stress Scale, a significant main effect for Condition F(1,54)=640.99, p<0.001 was evidenced. The main effect for Time, F(2,108)=2.51, p=0.08, and the Interaction were not significant, F(2,108)=0.69, p=0.50. Perceived stress decreased significantly from Baseline to Day 60 for DG only, p<0.001 (see FIG. 22). For cortisol levels, a significant main effect for Condition, F(1,54)=112.11, p<0.001, was evidenced. The main effect for Time, F(2,80)=0.02, p=0.98, and the Interaction, F(2,80)=0.02, p=0.98, were not significant. PG had higher Baseline and Day 60 cortisol levels compared to DG (see FIG. 50).

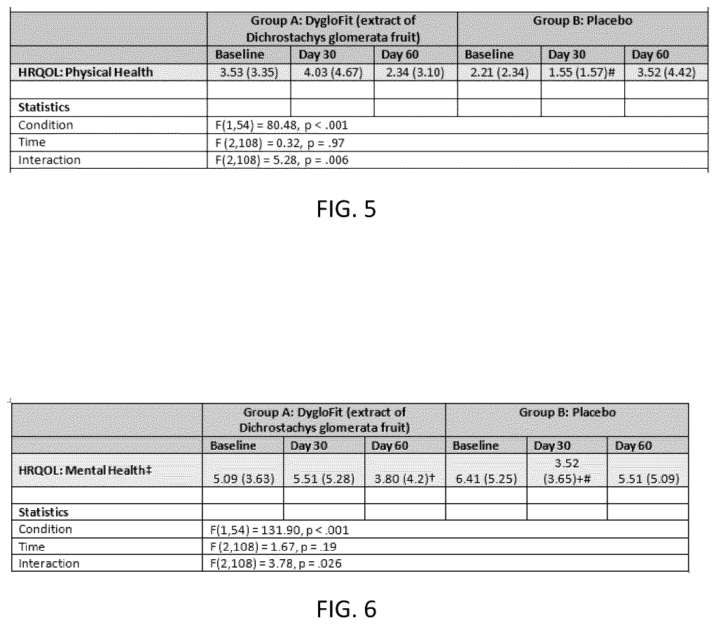

Health-related Quality of Life: For General Health, a significant main effect for Condition was evidenced, F(1,54)=2421.09, p<0.001, indicating an improvement in General Health for DG by Day 60 (see FIG. 4). The main effect for Time, F(2,108)=0.65, p=0.53, and the interaction, F(2,108)=0.12, p=0.89, were not significant (see FIG. 4). For Physical Health significant main effects for Condition, F(1,54)=80.48, p<0.001, and Interaction, F(2,108)=5.28, p=0.006, but no significant effect for Time was evidenced, p=0.97. Physical health scores improved significantly for DG when compared to PG at the Day 60 time point. DG physical health improved from Baseline to Day 60 compared to worsening for PG, p<0.05 (see FIG. 5). As well, for Mental Health significant main effects for condition, F(1,54)=131.90, p<0.001, and interaction, F(2,108)=3.78, p=0.02, were evidenced; but no significant time effect, F(2,108)=1.67, p=0.19, was found. DG mental health improved significantly from Baseline to Day 60 compared to PG, p<0.01. PG mental health improved significantly from Baseline to Day 30, p<0.05 (see FIG. 6).

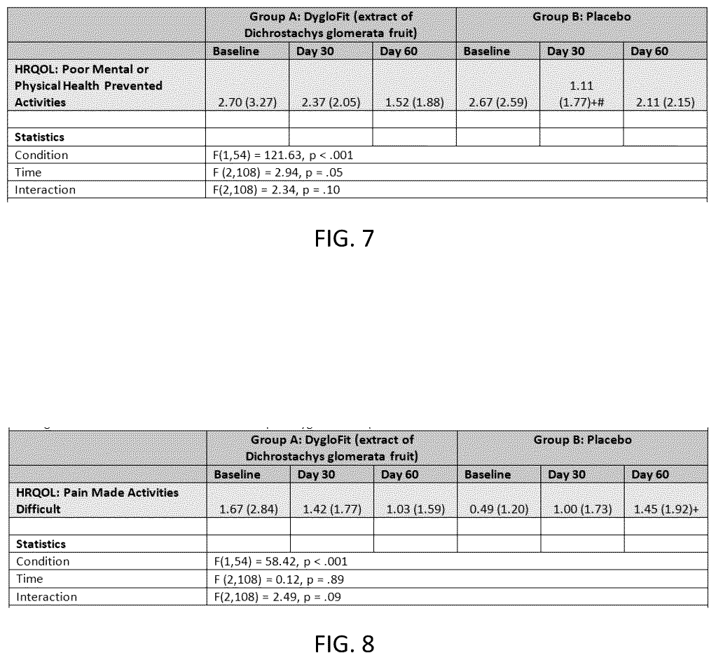

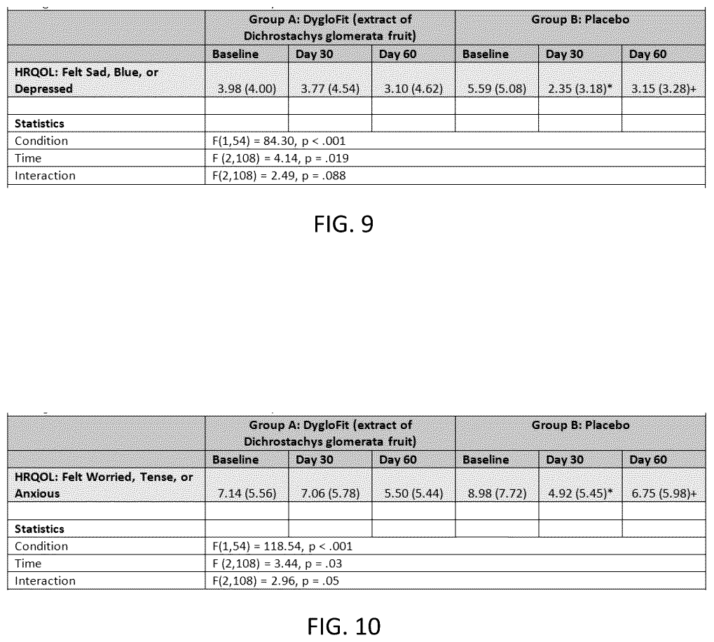

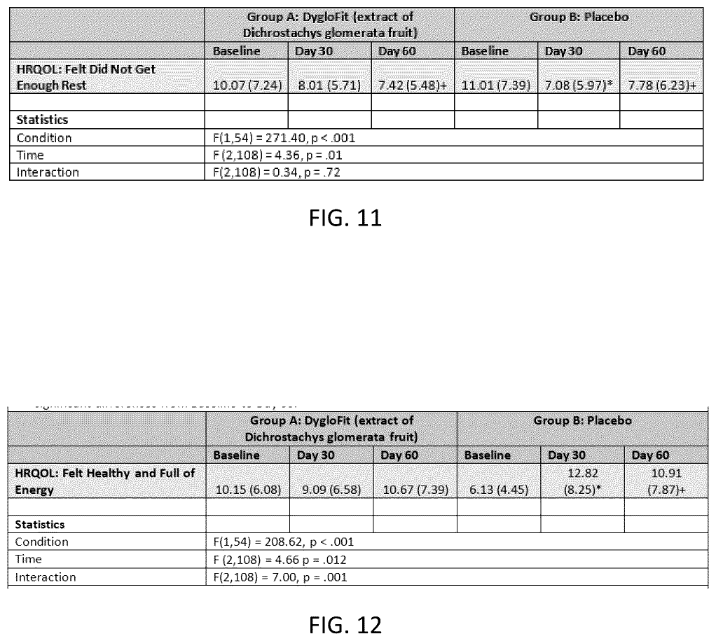

For poor mental or physical health preventing activities significant main effects for Condition, p<0.001, and Interaction, p=0.10, and Time, p=0.05, were evidenced for DG compared to PG. DG scores improved significantly compared to PG by Day 60, p<0.05. In comparison PG had a significant worsening in the number of days that pain made activities difficult from Baseline to Day 60, p<0.05 (see FIG. 7).

Moderator analysis by gender revealed no gender effects, p's >0.05.

### CONCLUSION

The results of the study shown and described herein, including the data shown and described in the figures, demonstrate that Dyglomera, a standardized powder derived from the aqueous ethanol extraction, concentration, and drying of Dichrostachys glomerata fruit pods, improved various health-related outcomes in overweight and mildly obese adults. Notably, Dyglomera has previously demonstrated anti-inflammatory properties and the capacity to regulate fat metabolism in both animal models and obese individuals with metabolic syndrome (Etoudi et al., 2010; Dieudonne et al., 2013; Kuae et al., 2010, 2011, 2013). Using a randomized double-blind placebo-controlled trial, we found that 60 days of supplementation with Dyglomera (hereafter, “DG”), resulted in, among other things, reduced anxiety, perceived stress, food cravings, stress and improved mood, HRQoL, physical health, mental health, energy and happiness that were statistically significant when compared to the placebo control group (hereafter, “PG”). Other quality of life factors also compared to the placebo control group.

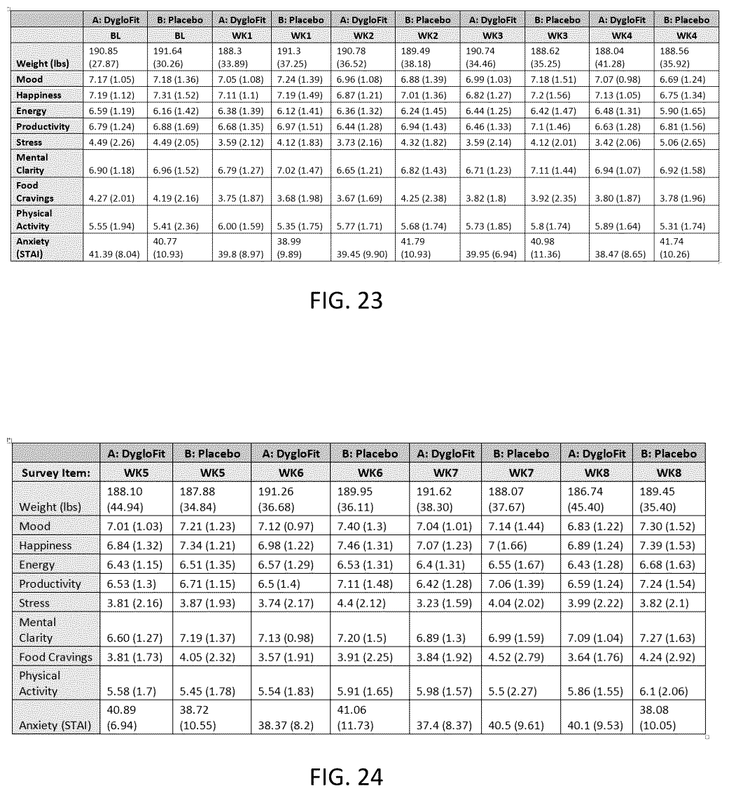

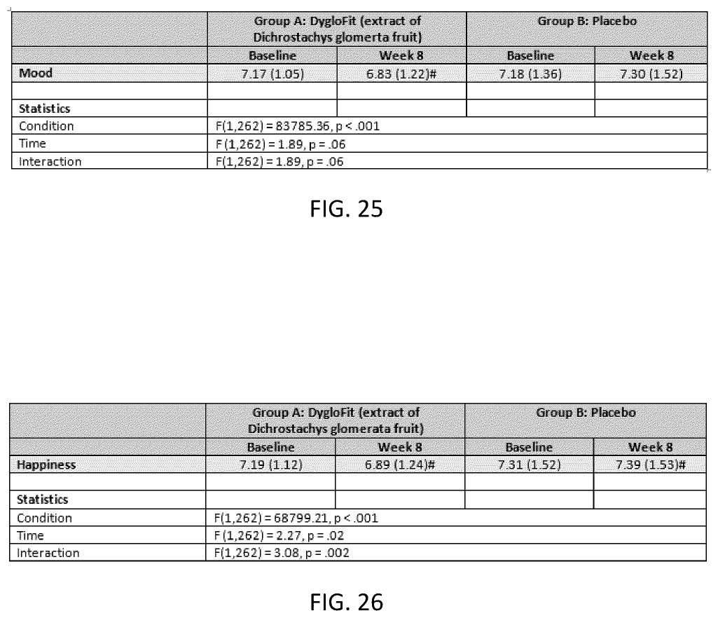

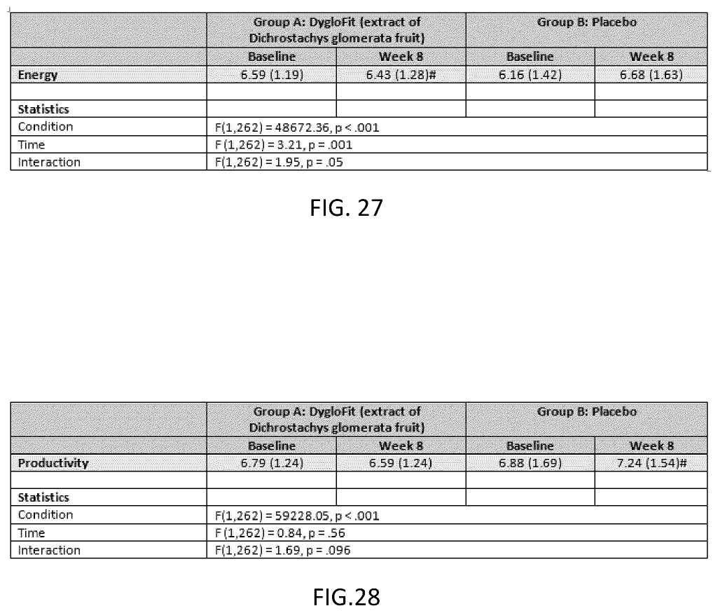

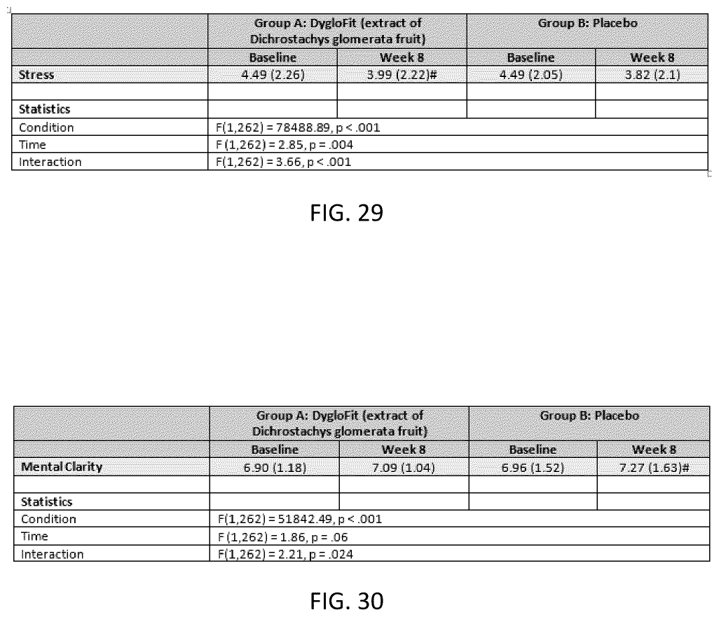

Regarding food cravings, DG also had significant reductions in food cravings from Baseline to Day 30 and Day 60 compared to PC (see FIGS. 23 and 24). Lab research found that Dyglomera (extract of Dichrostachys glomerata fruit) inhibited adipogenesis and lipogenesis by regulating AMPK phosphorylation in white adipose tissues (WATs) and 3T3-L1 adipocytes and promoted lipolysis by increasing the expression of lipolysis-related proteins (Kim et al 2022). These results suggest that Dyglomera (extract of Dichrostachys glomerata fruit) can be used as an effective dietary supplement for treating obesity due to its modulating effect on adipogenesis/lipogenesis and lipolysis. The results of the randomized controlled trial shown and described herein confirm that Dyglomera is an effective natural supplement to reduce anxiety and provide a variety of other health benefits.

DG had significant improvements compared to the PG by Day 60 for general health, physical health (which includes physical illness and injury), mental health (which includes stress, depression, and problems with emotions), and poor mental or physical health preventing activities such as self-care, work, or recreation. In comparison, PG had a significant worsening in the number of days that pain made activities difficult from Baseline to Day 60 (see FIG. 2).

For mood (POMS), DG mood improved significantly from Baseline to Day 60 compared to the PG. POMS measures the mood states of tension, esteem, anger, vigor, fatigue, depression, and confusion (see FIG. 20). Anxiety decreased significantly from Baseline to Day 60 for DG and from Baseline to Day 30 PG.

Perceived stress decreased significantly from Baseline to Day 60 for DG only. A significant reduction in perceived stress was observed in DG, indicating a potential stress-reduction effect associated with the consumption of Dyglomera (extract of Dichrostachys glomerata fruit) (see FIG. 22). It is noteworthy that the PG exhibited higher cortisol levels at both baseline and Day 60, highlighting a potential hormonal response (see FIG. 50).

The results and data obtained from the experiments described herein indicate that the safety and effectiveness of DG in all mammals, including but not limited to, overweight and mildly obese adults.

Consistent with animal and lab tests (Kothari) Dyglomera was found safe and well-tolerated with the participants. Only one minor adverse event was reported in DG (i.e., gastrointestinal issues). Results can be generalized to similar weight and age groups.

In summary, the study examined the various effects of Dichrostachys glomerata fruit pod powder (Dyglomera) on overweight and mildly obese adults in a double-blind, placebo-controlled trial. The results revealed that the DG experienced significant reduced food cravings, anxiety levels and stress, and improved mood, physical health, mental health, energy, happiness and improved other quality of life factors leading to an enhanced quality of life. Notably, the DG also showed a reduction in perceived stress, while the PG exhibited higher cortisol levels. These findings suggest the potential of Dichrostachys glomerata as a beneficial supplement for various conditions, including but not limited to anxiety and overall well-being.

### REFERENCES

Arterburn D E, Telem D A, Kushner R F, Courcoulas A P. Benefits and Risks of Bariatric Surgery in Adults: A Review. JAMA. 2020 September 1; 324(9): 879-887. doi: 10.1001/jama.2020.12567.
Azantsa B., Kuate D., Chakokam R., Paka G., Bartholomew B., Nash R. The effect of extracts of Irvingia gabonensis (IGOB131) and Dichrostachys glomerata (Dyglomera) on body weight and lipid parameters of healthy overweight participants. Funct. Foods Health Dis. 2015; 5: 200-208.
Batsis J A et al. A Systematic Review of Dietary Supplements and Alternative Therapies for Weight Loss. Obesity. 2022: 29(7): 1102-1113. doi: 10.1002/oby.23110
Bray G A, William E Heisel, Ashkan Afshin, Michael D Jensen, William H Dietz, Michael Long, Robert F Kushner, Stephen R Daniels, Thomas A Wadden, Adam G Tsai, Frank B Hu, John M Jakicic, Donna H Ryan, Bruce M Wolfe, Thomas H Inge. The Science of Obesity Management: An Endocrine Society Scientific Statement. Endocr Rev. 2018 Apr; 39(2): 79-132. Published online 2018 Mar. 6. doi: 10.1210/er.2017-00253
Centers for Disease Control and Prevention (CDCP). Health-related quality of life. [Accessed Oct. 1, 2020]. http://www.cdc.gov/hrqol.
Centers for Disease Control and Prevention (CDCP). Overweight & Obesity; 2016 [WWW document]. URL https://www.cdc.gov/obesity/ (accessed 5 Dec. 2023).
Centers for Disease Control and Prevention. Defining adult obesity. Updated Jun. 7, 2021. Accessed Dec. 5, 2023. www.cdc.gov/obesity/basics/adult-defining. html Cohen, S. Kamarck T. & Mermelstein, R. (1983). A global measure of perceived stress. Journal of Health and Social Behaviour, 24, 385-396.
Dalton M, Finlayson G, Walsh B, Halseth A E, Duarte C, Blundell J E. Early improvements in food cravings are associated with long-term weight loss success in a large clinical sample. Int J Obes (Lond). 2017 Aug; 41(8): 1232-1236. doi: 10.1038/ijo.2017.89.
Dieudonne K., Anne P. N. K., William D., Blanche C. O., Etoundi-Ghislain D. P. Effectiveness of Dichrostachys glomerata spice phenolics in reduction of oxidative stress associated with obesity and type 2 diabetes; a randomized, double-blind placebo-controlled clinical trial. J. Food Res. 2013; 2: 887-895.
Djuissi N. M., Ngoula F., Kouamo J., Vemo N. B., Nono M. F. S., Lontio A. F., Tchoffo H., Dongmo A. N. Reproductive Characteristics, Serum Metabolites, and Oxidative Status in Female Guinea Pigs (Cavia porcellus) Fed with Ethanolic Extract of Dichrostachys glomerata Fruit. World. 2021; 11: 66-72. doi: 10.54203/scil.2021.wvj9
Du L, Yong G, Wang P, Wang X, Ming W, He G. Developing the modified 4-item version of perceived stress scale for functional dyspepsia. BMC Gastroenterol. 2023 Mar. 29; 3(1): 97. doi: 10.1186/s12876-023-02728-0.
Etoundi C. B., Kuaté D., Ngondi J. L., Oben J. Anti-amylase, anti-lipase and antioxidant effects of aqueous extracts of some Cameroonian spices. J. Nat. Prod. 2010; 3: 17.
Fontaine, K. R., Barofsky, I., & Cheskin, L. J. (2003). Predictors of quality of life among obese persons. Journal of Psychosomatic Research, 54(4), 369-375.
Kim T N. Barriers to Obesity Management: Patient and Physician Factors. J Obes Metab Syndr. 2020 December 30; 29(4): 244-247. doi: 10.7570/jomes20124.
Kim H L, Lee S K, Min D E, Choi B K, Lee D R. Anti-Obesity Effect of Dyglomera® Is Associated with Activation of the AMPK Signaling Pathway in 3T3-L1 Adipocytes and Mice with High-Fat Diet-Induced Obesity. Molecules. 2022 May 20; 27(10): 3288. doi: 10.3390/molecules27103288.
Kolotkin R L, Andersen J R. A systematic review of reviews: exploring the relationship between obesity, weight loss and health-related quality of life. Clin Obes. 2017: 7(5): 273-289. doi: 10.1111/cob.12203.
Kothari S C, Shivarudraiah P, Venkataramaiah S B, Gavara S, Arumugam N, Soni M G. Toxicologic evaluation of Dichrostachys glomerata extract: subchronic study in rats and genotoxicity tests. Food Chem Toxicol. 2014 Jul: 69: 120-31. doi: 10.1016/j. fct.2014.03.045.
Krukowski R A, Ross K M. Measuring Weight with Electronic Scales in Clinical and Research Settings During the Coronavirus Disease 2019 Pandemic. Obesity (Silver Spring). 2020 Jul; 28(7): 1182-1183. doi: 10.1002/oby.22851.
Kuate D., Etoundi B. C. O., Soukontoua Y. B., Ngondi J. L., Oben J. E. Antioxidant characteristics of Dichrostachys glomerata spice extracts. CYTA J. Food. 2010; 8: 23-37. doi: 10.1080/19476330903129126.
Kuate D., Etoundi B.C., Ngondi J. L., Oben J. E. Effects of Dichrostachys glomerata spice on cardiovascular diseases risk factors in normoglycemic and type 2 diabetic obese volunteers. Food Res. Int. 2011; 44: 1197-1202. doi: 10.1016/j. foodres.2010.09.037.
Kuate D., Etoundi B. C., Ngondi J. L., Manan W. A., Muda B. W., Oben J. E. Anti-inflammatory, anthropometric and lipomodulatory effects Dyglomera(hydroethanolic extract of Dichrostachys glomerata) in obese patients with metabolic syndrome. Funct. Foods Health Dis. 2013; 3: 416-427. doi: 10.31989/ffhd. v3i11.35.
McNair, D. M., Lorr, M., & Droppleman, L. F. (1992). Manual of the Profile of Mood States. EdITS/Educational and Industrial Testing Service, San Diego, CA1992
Meule A, Hermann T, Kübler A. A short version of the Food Cravings Questionnaire-Trait: the FCQ-T-reduced. Front Psychol. 2014; 5(190): 1-10. doi: 10.3389/fpsyg.2014.00190.
Riaz H, Khan M S, Siddiqi T J, Usman M S, Shah N, Goyal A, Khan S S, Mookadam F, Krasuski R A, Ahmed H. Association Between Obesity and Cardiovascular Outcomes: A Systematic Review and Meta-analysis of Mendelian Randomization Studies. JAMA Netw Open. 2018 November 2; 1(7): e183788. doi: 10.1001/jamanetworkopen.2018.3788.
Ross K M, Wing R R. Concordance of In-Home “Smart” Scale Measurement with Body Weight Measured In-Person. Obes Sci Pract. 2016 Jun; 2(2): 224-248. doi: 10.1002/osp4.41.
Spielberger C, Gorsuch R, Lushene R, Vagg P and Jacobs G (1983) State-Trait Anxiety Inventory For Adults. Consulting Psychologists, Palo Alto.
Tchiégang C., Mbougueng P. D. Chemical composition of spices used in the cooking of nah poh and nkui of western Cameroon. Tropicultura. 2005; 23: 193-200.
Watanabe M, Risi R, Masi D, Caputi A, Balena A, Rossini G, Tuccinardi D, Mariani S, Basciani S, Manfrini S, Gnessi L, Lubrano C. Current Evidence to Propose Different Food Supplements for Weight Loss: A Comprehensive Review. Nutrients. 2020 September 20; 12(9): 2873. doi: 10.3390/nu1209287
Williamson, D. A., Rejeski, J., Lang, W., Van Dorsten, B., Fabricatore, A. N., & Toledo, K. (2000). Impact of a weight management program on health-related quality of life in overweight adults with type 2 diabetes. Archives of Internal Medicine, 160(12), 1777-1786.
Zack, M. M. (2013). Health-related quality of life—United States, 2006 and 2010. Morbidity and Mortality Weekly Report, 62, 105-111.

What is claimed is:

1. A composition for reducing food craving in a mammal, the composition comprising:
an effective amount of Dichrostachys glomerata;
wherein the composition is provided as an oral dosage unit in the form of a pill, capsule, liquid, lozenge or tablet.

2. The composition of claim 1, wherein the effective amount of Dichrostachys glomerata is 100 mg to 900 mg daily.

3. The composition of claim 1, wherein the Dichrostachys glomerata composition is an extract of Dichrostachys glomerata fruit.

4. The composition of claim 1, wherein the Dichrostachys glomerata composition is an ethanol extract of Dichrostachys glomerata.

5. The composition of claim 1, wherein the Dichrostachys glomerata composition is comprised of Dichrostachys glomerata fruit pods.

6. The composition of claim 1, wherein the Dichrostachys glomerata composition is comprised of dried Dichrostachys glomerata fruit pods.

7. The composition of claim 1, wherein the composition is provided to a mammal for at least 30 days.

8. A composition for reducing food cravings in a mammal, the composition comprising:
an effective amount of Dichrostachys glomerata fruit pods;
wherein the composition is provided as an oral dosage unit in the form of a pill, capsule, liquid, lozenge or tablet.

9. The composition of claim 8, wherein the effective amount of Dichrostachys glomerata is 100 mg to 900 mg daily.

10. The composition of claim 8, wherein the Dichrostachys glomerata composition is extract of Dichrostachys glomerata fruit.

11. The composition of claim 8, wherein the Dichrostachys glomerata composition is an ethanol extract of Dichrostachys glomerata.

12. The composition of claim 8, wherein the Dichrostachys glomerata composition is an aqueous ethanol extract of Dichrostachys glomerata.

13. The composition of claim 8, wherein the Dichrostachys glomerata composition is comprised of dried Dichrostachys glomerata fruit pods.

14. The composition of claim 8, wherein the composition is provided to a mammal for at least 30 days.

15. A method of reducing food cravings in a mammal, the method comprising:
providing a composition containing an effective amount of Dichrostachys glomerata to a mammal.

16. The method of claim 15, wherein the composition is provided as an oral dosage unit in the form of a pill, capsule, liquid, lozenge or tablet.

17. The method of claim 15, wherein the effective amount of Dichrostachys glomerata is 100 mg to 900 mg daily for at least 30 days.

18. The method of claim 16, wherein the Dichrostachys glomerata composition is an extract of Dichrostachys glomerata fruit.

19. The method of claim 16, wherein the Dichrostachys glomerata composition is an ethanol extract of Dichrostachys glomerata.

20. The method of claim 16, wherein the Dichrostachys glomerata composition is comprised of Dichrostachys glomerata fruit pods.
